(19) United States
(12) Patent Application Publication
(10) Pub. No.: US 2011/0257550 A1
(43) Pub. Date: Oct. 20, 2011

(54) Method and Apparatus for Continuous Monitoring of Exhaled Carbon Dioxide

(76) Inventor: Jay Choi, Bernardsville, NJ (US)

(21) Appl. No.: 13/051,288

(22) Filed: Mar. 18, 2011

Related U.S. Application Data
(60) Provisional application No. 61/340,611, filed on Mar. 20, 2010.

(51) Int. Cl. A61B 5/097

(52) U.S. Cl. 600/532

(57) ABSTRACT

The present invention provides for a unique modification of the intravenous catheter, attached to the terminal line of the mass spectrometer and the use of a disposable nasal oxygen cannula provide an effective and efficient port to monitor end tidal carbon dioxide (P_{ET}CO₂) in unintubated, conscious, spontaneously breathing patients who are receiving administration of local and regional anesthesia or during recovery from residual general anesthesia.

### CROSS REFERENCE TO RELATED APPLICATIONS

This application claims the benefit of priority pursuant to 35 U.S.C. §119(c) to U.S. Provisional Patent Application No. 61/340,611, filed on Mar. 20, 2010, which is incorporated by reference in its entirety herein.

### BACKGROUND OF THE INVENTION

Anesthesia allows patients to undergo surgery and other procedures without the distress and pain they would otherwise experience. During sedation, oxygen is administered to the patient via medical tubing connected to a nasal cannula. During surgery, patients being administered anesthetic drugs are continuously monitored to ensure the patient's safety. One of the standard monitoring tools is a capnograph, which monitors the carbon dioxide (CO₂) in the exhaled respiratory gases. Monitoring amount of CO₂ in exhaled respiratory gasses, which is known as capnography, provides one of the most rapid and reliable methods to detect life-threatening conditions, including movement of tracheal tubes, unsuspected ventilatory failure, circulatory failure and defective breathing circuits, and enables physician to circumvent potentially irreversible patient injury.

CO₂ is produced by cellular metabolism and is transported to the right heart by the venous system. It is then pumped into the lungs by the heart and then diffuses out into the exhaled air where it can be measured. End tidal carbon dioxide (P_{ET}CO₂), therefore, is a reflection of metabolism, circulation and ventilation. Specifically, P_{ET}CO₂ measures CO₂ at the very end of expiration. It is the maximum concentration of expired CO₂.

P_{ET} CO₂ monitoring allows exhaled CO₂ to be measured non-invasively. When a patient is being sedated or operated upon, it is necessary to supplement the inhalation with a treating gas, such as oxygen or a gaseous anesthetic. In these instances, an accurate quantitative determination of the amount of at least one gaseous component, such as carbon dioxide in the exhalation is highly desirable. In intensive care situations or under a regional or general anesthetic, an accurate determination of such compositions allows bodily functions of a patient to be readily monitored and treatment of the patient to be properly optimized accordingly. Measuring the concentration of carbon dioxide in exhaled breathing gas may be conducted continuously to provide short response times and to enable rapid alterations in an ongoing medical treatment strategy, thereby preventing and minimizing adverse effects or damage to the patient.

One area of particular interest is the monitoring of end-tidal carbon dioxide, which is the partial pressure of the carbon dioxide component of exhaled gas at the end of exhalation in a mechanically ventilated or spontaneously breathing patient. Even with supplemental oxygen administration and pulse oximetry monitored, the qualitative monitoring of end-tidal carbon dioxide is useful in spontaneously breathing patients who are unintubated while being sedated or awake during regional or local anesthesia being given, or post-operatively during emergence from residual general anesthesia.

Current devices for sampling of gaseous components in the exhalation during general anesthesia allow a quantitative as well as qualitative analysis of the carbon dioxide which correlates adequately with the actual amount of this gaseous component in the arterial blood. However, in spontaneously breathing patients, monitoring of end-tidal carbon dioxide has not been sufficiently reliable due to various reasons, such as technical difficulty, cost-effectiveness, and patient's discomfort.

Prior techniques for insufflating a treating gas into the breathing gas of a patient and simultaneously measuring at least one gaseous component of the exhalation of the patient have involved withdrawing a breathing gas sample through a chamber or conduit receiving both exhaled gas and at least some amount of the insufflated treating gas.

A nasal airway can be highly uncomfortable because it partially blocks and in some instances irritates the nasal passage of the patient. Although the modified nasal airway device produced allegedly a “satisfactory ET CO₂ curve”, it is not utilized routinely to sedated or awaken patients in general. In addition, there was no provision of a device for simultaneous insufflating a treating gas such as oxygen.

Although a sampling catheter so arranged may be used to monitor ventilatory exchange during regional anesthesia, those attempts were unsuccessful for quantitative measurements because of excessive mixing and erratic differences between measured values of end-tidal carbon dioxide and arterial carbon dioxide. This problem led other researchers in the field to try other approaches for quantitative measurements of end-tidal carbon dioxide in unintubated patients while administering supplemental oxygen.

There have been a few efforts of breath sampling from an awake patient receiving supplemental oxygen to quantitatively determine the magnitude of respiratory depression occurring as a result of local or regional anesthesia or intravenous sedation.

In order to obtain an undiluted end tidal gas sample, Salter (U.S. Pat. No. 5,137,017) disclosed a demand oxygen system with a separate end tidal sampling cannula, which is an intermittent oxygen delivery system for supplying oxygen to a first nare of a nasal cannula in response to patient's exhalation sensed through the second nare which is isolated from the delivery of the oxygen to the first nare. Salter provides a system and a method for controlling the flow of oxygen from a source of oxygen to a patient through a nasal cannula where the flow is controlled by valve means that are operated in response to the initiation of exhalation by the patient. The nasal cannula consists of a conventionally shaped nasal cannula face piece having inlet and outlet conduits communicating respectively with two separate zones in the face piece which are separated by a gas-tight partition means in the face piece. The nasal cannula is provided with two mires or tubes that terminate adjacent the patient's nostrils as is conventional. However, each mire communicates with different zones in the face piece. While this cannula claims obtaining an undiluted end tidal gas sample, it is heavy, bulky, hence adding more discomfort to patients and problems of cost-effectiveness.

Curti, et al (U.S. Pat. No. 6,439,234) disclosed an apparatus for insufflating a treating gas into a patient and for measuring a carbon dioxide content exhaled by the patient includes a hollow body with a partitioning wall separating the body into inhalation and exhalation manifolds. Two hollow prongs have coaxial openings close to the body which allow gas exchange for breathing and carbon dioxide measuring purposes. The holes are of a size such that suction drawn through the holes is limited while still allowing accurate gas analysis.

Bowe, et al (U.S. Pat. No. 5,335,656) disclosed a method and apparatus for inhalation of treating gas and sampling of exhaled gas for quantitative analysis, in which a nasal cannula is described for insufflating a treating gas into one nostril and measuring at least one gaseous component of exhaled breath in the other nostril of a living body. The cannula includes a wall member cooperating with a hollow body of the cannula to define inhalation and exhalation manifolds which engages the hollow body to provide a gas-tight seal for positive prevention of fluid communication between these manifolds. A hollow nasal prong communicates with each manifold and is positioned and shaped to be received in a corresponding nostril. The wall member may be integrally molded with the body of the cannula when it is made or a conventional cannula may be modified by insertion of a wall member to create separate manifolds.

All these efforts were concentrated excessively on the quantitative analysis of the magnitude of respiratory depression occurring as a result of local or regional anesthesia or intravenous sedation, placing less emphasis on the qualitative evidence of breathing with the patient and provider friendly device as well as being cost-effective.

There is therefore a need in the art for an insufflating and sampling apparatus having the combined advantageous of insufflating a treating gas into an awaken patient and sampling a portion of the patient's exhaled breathing gas in a maimer providing a reasonable degree of qualitative evidence of spontaneous breathing and comparable degree of quantitative correlation of a gaseous component in the breathing gas with or without simultaneous administration of oxygen. Prior art efforts were focused predominantly on the quantitative analysis of the magnitude of respiratory depression, while placing less emphasis on the qualitative evidence of spontaneous breathing, which is far greater significant in terms of sensitivity and specificity. The present invention satisfies the need in the art.

### SUMMARY OF THE INVENTION

The invention provides an apparatus for measuring carbon dioxide content in the exhaled air of a patient while simultaneously insufflating a treating gas into the nose of the patient. In one embodiment, the apparatus comprising a nasal cannula having two nares wherein the nasal cannula is modified to contain a catheter adapted to fit within one nave of the nasal cannula. Preferably, the catheter is connected into a gas sampling line wherein the sampling line is connected to a capnometer.

In one embodiment, the catheter is substantially less than about 5 mm in diameter. In another embodiment, the catheter is less than about 3 mm in diameter. In yet another embodiment, the catheter is less that about 2 mm in diameter. In another embodiment, the catheter is about 1.7 mm in diameter.

In one embodiment, the catheter is about 18 mm long and is about 1.7 mm in diameter, further wherein the catheter contains a stylet at one end and a beveled tip at the other end thereby forming a sampling CO₂ port.

In one embodiment, the catheter is adapted to fit within one nare of the nasal cannula, further wherein the proximal end of the sampling CO₂ port is connected into a gas sampling line wherein the sampling line is connected to a capnometer.

The invention provides a method for monitoring end tidal CO₂ in a patient comprising providing a nasal cannula to a patient wherein the nasal cannula is modified to contain catheter adapted to fit within one nare of the nasal cannula, further wherein the catheter is connected into a gas sampling line wherein the sampling line is connected to a capnometer.

The invention provides an apparatus comprising a catheter having a tapered end and a housing component. Preferably, the apparatus further comprises a stylet.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 shows a sampling CO₂ port 100 with stylet 120 in place, having a hard plastic catheter 110. The stylet is without an additional safety feature, which is necessary only for an intravenous catheter.

FIG. 2 shows two units that make up the sampling CO₂ port 100 of FIG. 1. Unit 200 shows a stylet comprising a handle 220 and a needle 210. The needle has a point 230. Unit 250 comprises a catheter having a tapered end towards the end of the catheter 260 and a housing component 270.

FIG. 3 shows a cannula face piece 320 having two nasal prongs 330. The CO₂ port with stylet 300 in place having a needle point 315 can be used to pierce the cannula face piece 320. FIG. 3A shows the diameter of a representative catheter 310 used for an adult. FIG. 3B shows the diameter of a representative catheter 310 used for a child/infant.

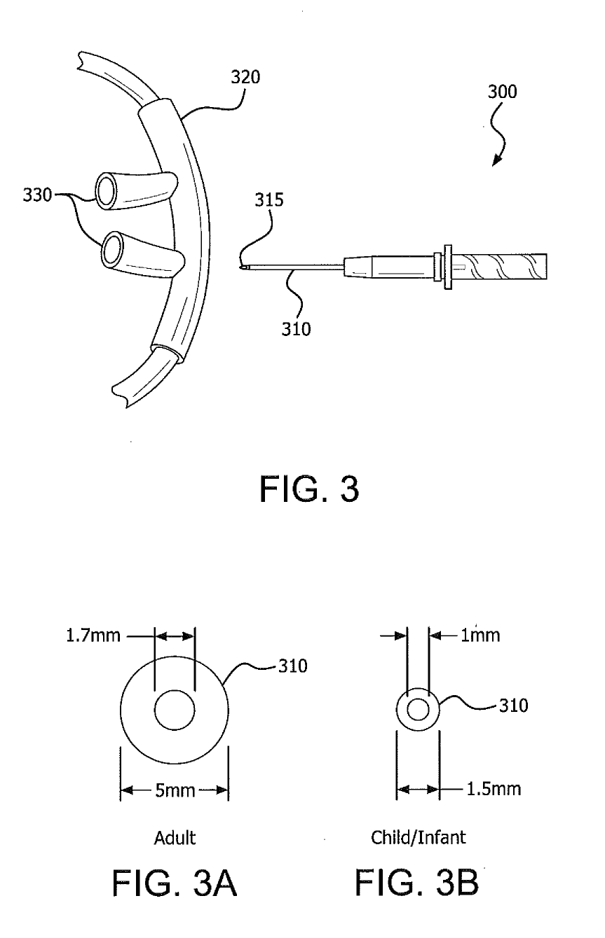

FIG. 4 shows the position of the sampling CO₂ port 400 within the nasal prong of a cannula 430 at a distance shorter than the end of the nasal prong 440. The CO₂ port 400 is adapted to fit within a corresponding nasal prong of about 20 mm long, which the cannula face piece 430 is to be placed into the nasal passage of a patient. The proximal end of the sampling CO₂ port 400 is twisted into a gas sampling line using a Luer lock 410, which is then connected to the capnometer 420.

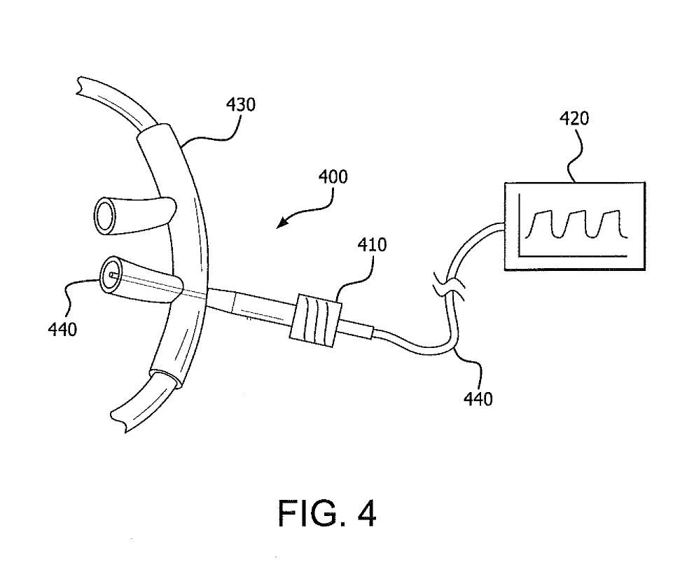

### DETAILED DESCRIPTION

This invention relates to an apparatus and a method for collecting and analyzing gases respired by a patient. The invention further is useful for capnographic and capnometric analysis for the content of carbon dioxide (CO₂) in the gases respired by a patient while simultaneously insufflating a treating gas, such as oxygen, into the inhalation of the patient. Capnometry is the measurement of expired CO₂ and provides a numeric display of CO₂ tension in mm Hg or % CO₂. Capnography is the graphic representation of expired CO₂ over time. Capnometer/Capnograph is the measuring instrument. Capnogram is the waveform displayed by the capnograph.

The present invention addresses the need to provide more reliable end-tidal gas values. It should be noted that while most of the present discussion takes place with reference to CO₂, the methods described herein apply to other gases as well, including but not limited to respiratory gases, such as oxygen, nitrous oxide, and other gases, such as anesthetic agents. To determine a more reliable end-tidal gas value, it is important to delineate properly the end-tidal gas value and to determine the reliability of that estimate.

The present invention is based on the fact that expired carbon dioxide monitoring is valuable to diagnose a simple presence or absence of respirations, airway obstruction, or respiratory depression. Mechanisms of End-tidal CO₂ detection according to the present invention provide a high level of sensitivity and specificity in the patient with spontaneous circulation.

Monitoring end tidal CO₂ according to the invention provides various benefits. For example, it allows end tidal sampling with uninterrupted simultaneous oxygen delivery. In one embodiment, end tidal CO₂ monitoring according to the invention provides positive qualitative and expected quantitative readings just as seen during general anesthesia. The methods and procedures of the present invention are safe, certain and cost-effective. The ease and comfort for providing such diagnostic measurements as contemplated herein are desirable because the procedure is light-weight, does not have an irritating flap or ridge, and is user-friendly to the practitioner.

A further benefit of the method and apparatus of the present invention is that it does not interfere with patient observation by medical practitioners. The present invention for the method and apparatus for continuous monitoring of exhaled carbon dioxide is versatile and compatible with all currently available nasal cannulas and modalities prescribed. One advantage of the present invention is the simplicity, ease, but effective device for continual monitoring of the presence of expired carbon dioxide, hence monitoring of spontaneous breathing.

In one embodiment, the present invention enables the ease for switching to general endotracheal anesthesia because the gas sampling line can be quickly reattached to the elbow port of the Breathing circuit.

The present invention includes a disposable nasal cannula which provides an effective and efficient port to monitor P_{ET}CO₂. In one embodiment, the measurement of P_{ET}CO₂ can be in an unintubated, conscious, spontaneously breathing patient who is receiving local and/or regional anesthesia. The invention is also applicable to a patient during recovery from residual general anesthesia.

Analysis of P_{ET}CO₂ and other exhaled gases has been a standard anesthesia practice in intubated patients and recommended in patients of non-intubated deep sedation. However, P_{ET}CO₂ monitoring of non-intubated patients is often limited due to various practical, technical issues as well as a cost-effective reason. Supplemental oxygen, delivered by mask or conventional nasal cannula, tends to dilute end tidal gases and distort waveforms. The present invention is also designed for end-tidal sampling in patients. Preferably, the patient is of non-intubated, moderate to deep sedation whom requires supplemental oxygen via nasal prongs. The invention allows for spontaneous breathing with the presence of end tidal CO₂ in exhaled air and allows for a quantitative analysis even with simultaneous insufflations of oxygen being provided. In one embodiment, the invention allows for an analysis of P_{ET}CO₂ that is comparable to those obtained in intubated patients.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “body fluids” includes any fluids which can be obtained from a mammalian body. Thus, the term “body fluids” also includes homogenates of any tissues and other body matter. More particularly, however, the term “body fluids” includes fluids that are normally or abnormally secreted by or excreted from the body. The respective fluids may include, but are not limited to: blood, plasma, lymph, urine, and cerebrospinal fluid, blood, plasma, and cerebrospinal fluid.

The term “capnometry” as used herein refers to the measurement of expired CO₂ and provides a numeric display of CO₂ tension in mm Hg or % CO₂.

The term “capnography” as used herein refers to the graphic representation of expired CO2 over time. Capnograph is the measuring instrument. Capnogram is the waveform displayed by the capnograph.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

The term “end-tidal CO₂ (ETCO₂)” as used herein refers to the measurement of CO₂ at the very end of expiration. It is the maximum concentration of expired CO₂.

The term “PaCO₂” is used herein to refer to the partial pressure of CO₂ in arterial blood.

As used herein, an “instructional material” includes a publication, a recording, a diagram, or any other medium of expression which can be used to communicate the usefulness of the apparatus of the invention. The instructional material of the apparatus of the invention can, for example, be affixed to a container which contains the apparatus of the invention or be shipped together with a container which contains the apparatus. Alternatively, the instructional material can be shipped separately from the container with the intention that the instructional material and the apparatus be used cooperatively by the recipient.

The terms “medicinal gas”, “supplied gas”, “provided gas”, “delivered gas” and any variation or permutation thereof, as referred to herein, may refer to any gas which may be provided to a patient for medical purposes. For example, the medicinal gas may be oxygen (O₂), oxygen-enriched air and/or the like (wherein oxygen-enriched air is also hereinafter referred to simply as “oxygen”). In some embodiments, the medicinal gas may be humidified and/or warmed.

The terms “measurement”, “concentration”, “concentration measurement” and any variation or permutation thereof, as referred to herein in relation to sampling of exhaled breath, may refer to a measurement of concentration of one or more particular components, sometimes gaseous, in an exhaled breath. For example, a concentration of CO₂ and/or oxygen may be measured.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, “phenotypically distinct” is used to describe organisms, cells or components thereof, which can be distinguished by one or more characteristics, observable and/or detectable by current technologies. Each of such characteristics may also be defined as a parameter contributing to the definition of the phenotype. Wherein a phenotype is defined by one or more parameters an organism that does not conform to one or more of the parameters shall be defined to be distinct or distinguishable from organisms of the said phenotype.

A “prophylactic” treatment is a treatment administered to a subject who does not exhibit signs of a disease or exhibits only early signs of the disease for the purpose of decreasing the risk of developing pathology associated with the disease.

The term “sampling” and any variation thereof, as referred to herein, may refer to a suction force applied by a pump in order to draw gas (at times constituting a patient's exhaled breath), through a cannula, towards a device adapted to measure a concentration of one or more particular components, sometimes gaseous, in the drawn gas.

A “therapeutic” treatment is a treatment administered to a subject who exhibits signs of pathology, for the purpose of diminishing or eliminating those signs.

The term to “treat,” as used herein, means reducing the frequency with which symptoms are experienced by a patient or subject or administering an agent or compound to reduce the frequency with which symptoms are experienced.

As used herein, “treating a disease or disorder” means reducing the frequency with which a symptom of the disease or disorder is experienced by a patient. Disease and disorder are used interchangeably herein.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based on the discovery that end tidal CO₂ can be monitored in sedated patients in a simple and cost effective manner. Sedation refers to the use of pharmacological or non-pharmacological means to depress the central nervous system, provide analgesia, and reduce patient anxiety. The depth of sedation is not clearly divided into stages but rather refers to a therapeutic continuum ranging from minimal anxiolysis to coma.

Sedation may cause profound respiratory depression and hypoventilation. Thus, accurate monitoring of ventilator status of sedated patients is desirable. Pulse oximetry primarily has been used to assess oxygenation, but not ventilation. A decline in oxygen saturation (SpO₂) during room-air breathing is believed to be a reliable indicator of ventilator abnormalities, but the presence of such abnormalities goes undetected in the presence of supplemental oxygen.

Hypoventilation events often occur during procedural sedation. Capnography identifies these events earlier than oximetry. Capnography serves as an early warning device of an impending hypoxia. Therefore, measuring end tidal CO₂ in patients is desirable.

All patients receiving moderate sedation should receive supplemental oxygen through nasal prongs or by mask, and must be continuously monitored to assess the depth of sedation by pulse oximetry. However, ventilation and oxygenation are separate though related physiological processes. Hence monitoring oxygenation is not a substitute for monitoring ventilatory function. Capnography is a logical device to monitor ventilation during procedural sedation, although it has limitations, particularly in nasal sampling.

Deeply sedated patients can hypoventilate and become significantly hypercapnic without becoming hypoxic if they are given supplemental oxygen. End-tidal CO₂ monitoring may detect respiratory, depression sooner than pulse oximetry. In the majority of clinical cases, the respiratory obstruction occurs well before the onset of hypoxia. Therefore monitoring of respiratory status is essential to enable corrective measures before the occurrence of hypoxia. Capnography has evolved into a standard of monitoring during anesthesia because it has proven itself to be a valuable tool on recognizing ventilatory and circulatory events that could potentially lead to deleterious effects. One of the greatest assets of capnography is that it can identify situations that can potentially result in hypoxia. Hypoventilation can be detected reliably by pulse oximetry only when patients breathe room air. In patients with spontaneous ventilation, supplemental oxygen often masks the ability to detect abnormalities in respiratory function.

In short, expired carbon dioxide monitoring is valuable to diagnose a simple presence or absence of respirations, airway obstruction, or respiratory depression. End-tidal CO₂ detection approaches 100% sensitivity and specificity in the patient with spontaneous circulation.

In using this invention, it is important to be cognizant of factors which may affect the accuracy of monitoring end-tidal carbon dioxide. For example, accurate monitoring of end-tidal carbon dioxide while insufflating oxygen into the same nostril may not be feasible even with the presence of an intact nasal septum inside the cannula. However, the volume of exhalation far exceeds the volume of mixing of insufflated oxygen with exhaled gases at the nare. For this reason, whether the tip of the sampling catheter is within the prong coaxially or outside of prong separately, the observed P_{ET}CO₂ values are reasonably acceptable within close range. Obviously, obstruction of the nose or sampling line with bodily secretions may lead to spuriously low measurements. Another source of potential error is the occurrence of predominantly mouth breathing. Certain pathological conditions, such as pulmonary embolism, also may decrease P_{ET}CO₂, regardless of the sampling device used.

It also should be recognized that administration of a given flow of oxygen through two nostrils instead of one nostril may enhance a safety margin in case of the line obstruction from bending due to positional changes such as prone or lateral position without significant interference of quantitative value.

The practice of measuring end-tidal carbon dioxide during the administration of anesthesia, particularly regional anesthesia, is common in the art. The reasons that anesthesiologists have embraced this technique are described more fully in U.S. Pat. No. 5,335,656 which is incorporated herein by reference in its entirety. However, the present invention is based on an improvement on the prior art methods partly based on the simplicity and cost effective method of measuring end-tidal carbon dioxide.

The preferred nasal cannula used in this procedure is a cannula which insufflates the patient with oxygen through one nare of a cannula and separately samples the exhaled gases by drawing the exhaled gas from the other nare into a conventional carbon dioxide analyzer. The cannula is preferably provided with an internal wall, a partition, a barrier or a system in the face piece to keep the conduits completely separate from one another for insufflation and sampling, however, separate lines can be used or even multiple nares for insufflation and sampling, though the latter device substantially increases the risk of gases mixing which can distort the readings for end-tidal carbon dioxide. It is preferred that two nares only are employed and that each nare performs only one function, i.e., insufflation or sampling into or from separate nostrils, detecting pressure or breathing characteristics, etc. Likewise, insufflation has normally been continuous, however, it could advantageously be intermittent which would further improve the end-tidal carbon dioxide measurement by insuring that gases being sampled where representative of exhaled gases undiluted by the other gases being insufflated. Most preferably, the intermittent insufflation is accomplished by the apparatus and method described in U.S. Pat. No. 5,626,131 and U.S. Pat. Appln. Pub. No. 20050284484 which are incorporated herein by reference in their entirety.

The gas interface of the invention comprises the following:
a) Detach the gas sampling line (297 cm) from the patient end of the anesthesia breathing circuit and attach the other end to the monitoring unit of the capnometer, either stand-alone; bedside or transport capnograph or built-in anesthesia machine.
b) Insert the CO₂ Port of the present invention into either side of the nasal prong, and withdraw the stylet.
c) Attach the gas sampling line to the CO₂ Port,
d) Tighten all connections to Luer adapters with a push-twist motion. Insure that all connections are secure.
e) Check the test interface for proper capillary connections and function prior to use, then verify the end tidal CO₂ value in exhaled air and a waveform on monitor.
f) Replace sampling line if blockage or leakage is found when tested.

### Apparatus

In one embodiment, the invention provides a nasal cannula structure for sampling carbon dioxide. The nasal cannula structure of the invention reduces or eliminates the incidence of occlusion of the tip of the carbon dioxide sampling nare during the removal of carbon dioxide by the sampling line connected to a monitoring device and/or a source of suction or vacuum. Accordingly, the invention provides a simplified system for providing an intermittent flow of oxygen to a patient during supplemental oxygen therapy.

In one embodiment, the invention provides a nasal cannula for insufflating a patient with oxygen while accurately monitoring end-tidal carbon dioxide. Preferably, the nasal cannula functions properly for its intended purpose even when either or both nares become occluded for any reason. Therefore, the invention provides a minimum risk of distorting the end-tidal carbon dioxide readings from the sampled exhalation gases during the administration of anesthesia.

In one embodiment, the invention provides an end tidal CO₂ sampling catheter that is adapted for allowing an insufflation of oxygen and sampling of end tidal CO₂ with quick and easy catheter into the nasal cannula. Preferably, the insufflation of oxygen and sampling of end tidal CO₂ is opposite to either nare at a location proximate to the entrance of the nasal passage when the cannula is in use.

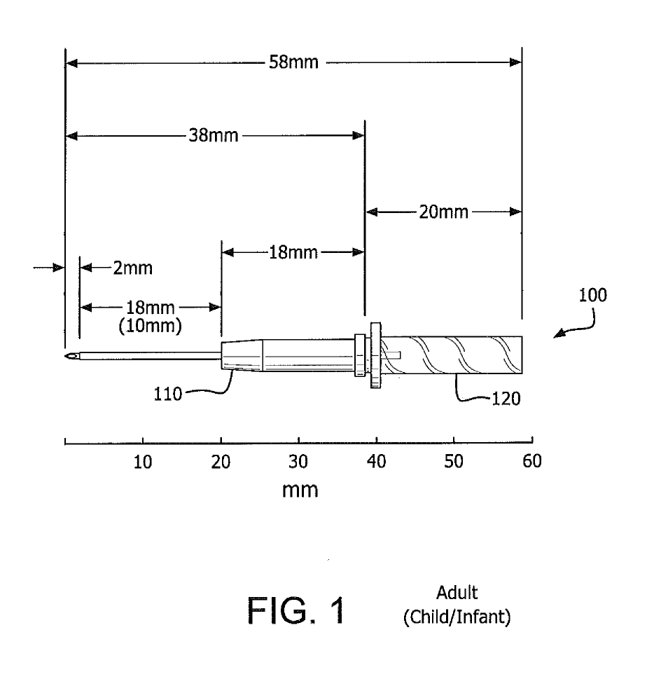

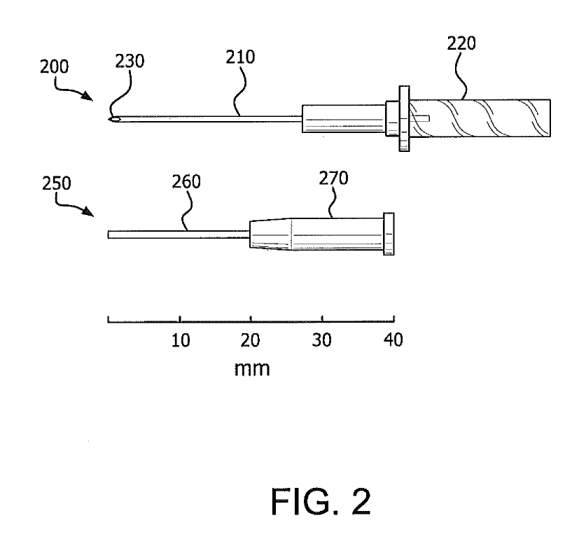

In one embodiment, the invention provides a sampling CO₂ port 100 depicted in FIG. 1. In one embodiment, the CO₂ port 100 comprises two units. The first unit is considered a functional unit. An example of such a functional unit is unit 250 depicted in FIG. 2. Preferably, unit 250 comprises a hard plastic catheter 260 and a housing component 270. Preferably, the rigidity of the hard plastic catheter 260 is substantially similar to the rigidity of housing component 270. The second unit can be considered the assisting unit. An example of such an assisting unit is unit 200. One method of making sampling CO₂ port 100 is to insert unit 200 into unit 250 by holding handle 220 and inserting the needle point 230 through housing unit 270 and catheter 260. The result is that the needle point 230 extends through the tip of the catheter 260.

In one embodiment, when the CO₂ port 100 of the invention is used for an adult, the catheter 260 is about 18 mm. In this situation, it is desirable to have the needle 210 length to be 20 mm so that the needle point 230 extends outside of catheter 260 about 2 mm. In some instances, the opening of the catheter 260 or 310 is about 1.7 mm.

When the CO₂ port 100 of the invention is used for a child or infant, the catheter 260 is about 10 mm. In this situation, it is desirable to have the needle 210 length to be 12 mm so that the needle point 230 extends outside of catheter 260 about 2 mm. In some instances, the opening of the catheter 260 or 310 is about 1 mm.

In one embodiment, the CO₂ port 100 of the invention can be inserted into a cannula face piece 320 by piercing the cannula face piece through one nasal prong 330. Once the port is inside the nasal prong, the assisting unit 200 can be withdrawn leaving only the functional unit 250 inside the nasal prong. A gas sampling line 440 can then be attached to the functional unit 250 by way of a Luer adapter or lock 410. The gas sampling line 440 can be connected to a capnometer 420.

In one embodiment, the invention provides a sampling port 100 for sampling end tidal CO₂ while allowing simultaneous administration of treating gas insufflation into an awake or sedated patient. The sampling port also provides accurate and reliable qualitative measurements of a gaseous component in exhaled breathing gas. Therefore, the sampling port also is useful in monitoring spontaneous breathing. The sampling port of the invention also provides a patient and provider friendly device to sample and measure end tidal CO₂ with technical ease, while being sensitive enough to monitor a positive evidence of spontaneous breathing. The sampling port of the invention also reduces or eliminates the incidence of occlusion of the tip of the carbon dioxide sampling compared to prior art apparatuses.

In one embodiment, the gas interface of the invention comprises the following: a) a gas sampling line that runs from the patient end of the anesthesia breathing circuit to the monitoring unit of the capnometer; b) the CO₂ port of the present invention inserted into either side of the nasal prong; e) the gas sampling line attached to the CO₂ Port.

In one embodiment, the catheter 260 is substantially less than about 5 mm in diameter, preferably less than about 4 mm in diameter, more preferably less than about 3 mm in diameter, yet more preferably less that about 2 mm in diameter. Most preferably, the catheter is about 1.7 mm in diameter.

In one embodiment, the invention encompasses a specially designed catheter 260 of about 16 gauge diameter with a stylet 220 that is inserted into one of the nasal prongs of the cannula, and connected to the distal end of the CO₂ sampling line which can be part of the breathing circuit. The catheter 260 itself does not occlude the terminal lumen of the prong 330 and therefore allows mixture of inspired oxygen. This monitoring technique provides reliable qualitative evidence of the presence of expired carbon dioxide. Presence of expired carbon dioxide represents evidence of spontaneous breathing and quantitative analysis.

It is, therefore, an embodiment of the present invention to provide a simplified system for providing a continuous flow of oxygen to a patient during supplemental oxygen therapy.

It is also an embodiment of the present invention to utilize common nasal cannula for insufflating a patient with oxygen while accurately monitoring end-tidal carbon dioxide that will continue to function properly for its intended purpose when either prong becomes occluded for any reason.

It is a further embodiment of the present invention to provide with an end tidal CO₂ sampling port as close to the end of prongs to minimize a mixing with a flow of oxygen, hence able to obtain an accurate end-tidal carbon dioxide reading from the sampled exhalation gases during the administration of anesthesia.

### Method

The present invention relates to methods for sampling exhaled breath from a patient. In some instances, the patient is simultaneously being provided with a medicinal gas. Medicinal gasses are often supplied to patients to treat respiratory and/or other medical problems. Sampling of exhaled breath of such patients and measuring concentration of one or more components in the sample may be beneficial in assessing the patient's medical condition via factors such as ventilation, perfusion, metabolism and/or the like.

In some instances, the provided medicinal gas may dilute the patient's exhaled breath, thereby biasing the measurement of concentration in the sampled exhaled breath. That is, if an outlet of a medicinal gas supply cannula is positioned in the vicinity of an inlet of an exhaled breath sampling cannula, some of the supplied medicinal gas may penetrate into the sampling cannula and thus bias the measurement. The present invention provides a method for overcoming this deficiency in the art by providing a more simple and accurate method of sampling and measuring exhaled breath from a patient.

The method of the invention can be performed with ease by uniquely modifying commonplace equipment and materials used in anesthesia. Given the ease of modifying existing cannula according to the present invention, the invention provides a useful tool in modern environment with an emphasis of patient comfort, safety and user-friendly speed of turnover as well as cost-effective alternatives. This simple arrangement allows simultaneous oxygen supplementation and continuous monitoring of ventilatory patterns and exchange.

In an aspect of the invention, the invention provides a method of evaluating and measuring CO₂ content at end expiration to obtain the end tidal partial pressure of exhaled CO₂ in the patient, wherein the measurement is made through the nostral. In one embodiment, a capnometer may include a gas capture member, for collecting expired gases, and a CO₂ measuring device attached to the gas capture member for determining levels of expired CO₂ from patient. In some instances, CO₂ levels may be measured continuously.

The method of the invention can be applied to standard respiratory gas monitoring systems. Such standard systems typically comprise gas sensing, measurement, processing, communication, and display functions. Such systems are considered to be either diverting (i.e., sidestream) or non-diverting (i.e., mainstream). A diverting gas measurement system transports a portion of the sampled gases from the sampling site, which is typically a breathing circuit or the patient's airway, through a sampling tube, to the gas sensor where the constituents of the gas are measured. A non-diverting gas measurement system does not transport gas away from the breathing circuit or airway, but measures the gas constituents passing through the breathing circuit.

Further, the end tidal CO₂ sampling catheter of the present invention is capable of administering oxygen to an awake patient while providing measurements of end-tidal carbon dioxide which are quantitatively equivalent to measurements of end-tidal carbon dioxide obtained by sampling via the separate Salter type cannula in a sedated or anesthetized patient.

All previous efforts were focused predominantly on the quantitative analysis of the magnitude of respiratory depression occurring as a result of local or regional anesthesia or intravenous sedation at the expense of practical issues such as patient safety and provider friendly device as well as being cost-effective, while placing less emphasis on the qualitative evidence of spontaneous breathing, which is far greater significant in terms of sensitivity and specificity.

Therefore, the invention provides a simple and cost effective method for insufflating and sampling end tidal CO₂ in an awaken patient. The method comprises using the apparatus of the invention which possesses the combined advantage of insufflating a treating gas into an awake patient and sampling a portion of the patient's exhaled breathing gas with a reasonable degree of qualitative evidence of spontaneous breathing and comparable degree of quantitative correlation of a gaseous component in the breathing gas with or without simultaneous administration of oxygen.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the apparatus of the present invention and practice the claimed methods. The following working examples therefore, specifically point out the preferred embodiments of the present invention, and are not to be construed as limiting in any way the remainder of the disclosure.

### Example 1

### Experimental Measurements and Comparisons

The following experiments were designed and performed as described. Briefly, the CO₂ port of the apparatus of the present invention was inserted into either side of the nasal prongs, and the stylet was withdrawn. The proximal end of the gas sampling line of about 297 cm was attached to the CO₂ port. The distal end of the sampling line from the Adult Circle Breathing Circuit, Vital signs Inc. was attached to Luer adapters of the infrared capnometer, Datex-Ohmeda Modulus 2 with a push-twist motion, then the end tidal CO₂ value and a waveform on monitor in exhaled air was verified. Calibration of the capnometer was performed according to the manufacturer's recommendations using a known sample gas containing 5% carbon dioxide, 40% nitrous oxide and 55% oxygen.

This demonstrated that the range of ET CO₂ was about 35-38 mm Hg without supplemental oxygen and 28-32 mm Hg with oxygen insufflated at a flow rate of 3 liters per minute in an awaken patient using Hudson RCI, Nasal Cannula with Flared Nasal Tips. Toward the end of expiration, the value of CO₂ reaches 32 mm Hg, which reflects the normal end-tidal value fairly well under most conditions.

Using Salter Labs, Ref 4002, Nasal Cannula (Adult) Salter Style, the range of ET CO₂ was 35-38 mm Hg without supplemental oxygen and 29-34 mm Hg with 3 liters per minute oxygen.

This data indicated the dilutional effect of supplemental oxygen around the coaxial CO₂ port appeared insignificant, which was only 1-2 mmHg lower than the Salter type cannula.

This data also demonstrated that measurements of end-tidal carbon dioxide with the cannula of this invention were compared favorably with the Salter type cannula of separate entry, and that the differences were insignificant. This data highlights the value of a quantitative measurement using a simple device of the present invention. It was unexpected that such a simple device of the invention would provide both desirable quantitative and qualitative measurements.

Accordingly, the CO₂ port provided accurate, consistent and reproducible measurements of the quantities of end-tidal carbon dioxide as a positive evidence of presence of spontaneous breathing, as well as a reasonable approximation of quantitative measurement of carbon dioxide. The CO₂ port of the present invention may be used for this purpose in awaken or sedated patients.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An apparatus for measuring carbon dioxide content in the exhaled air of a patient while simultaneously insufflating a treating gas into the nose of the patient, the apparatus comprising:
a nasal cannula having two nares wherein the nasal cannula is modified to contain a catheter adapted to fit within one nare of the nasal cannula, further wherein the catheter is connected into a gas sampling line wherein the sampling line is connected to a capnometer.

2. The apparatus of claim 1 wherein the catheter is substantially less than about 5 mm in diameter.

3. The apparatus of claim 1 wherein the catheter is less than about 3 mm in diameter.

4. The apparatus of claim 1 wherein the catheter is less that about 2 mm in diameter.

5. The apparatus of claim 1 wherein the catheter is about 1.7 mm in diameter.

6. The apparatus of claim 1, wherein the catheter is about 18 mm long and is about 1.7 mm in diameter, further wherein the catheter contains a stylet at one end and a beveled tip at the other end thereby forming a sampling CO₂ port.

7. The apparatus of claim 6, wherein the catheter is adapted to fit within one nare of the nasal cannula, further wherein the proximal end of the sampling CO₂ port is connected into a gas sampling line wherein the sampling line is connected to a capnometer.

8. A method for monitoring end tidal CO₂ in a patient comprising providing a nasal cannula to a patient wherein the nasal cannula is modified to contain catheter adapted to fit within one nare of the nasal cannula, further wherein the catheter is connected into a gas sampling line wherein the sampling line is connected to a capnometer.

9. An apparatus comprising a catheter having a tapered end and a housing component.

10. The apparatus of claim 9 further comprising a stylet.
